(19) United States
(12) Patent Application Publication
(10) Pub. No.: US 2011/0067707 A1
(43) Pub. Date: Mar. 24, 2011

(54) Lubricated Condom

(76) Inventor: John Shun Hui, Long Beach, CA (US)

(21) Appl. No.: 12/842,252

(22) Filed: Jul. 23, 2010

Related U.S. Application Data
(63) Continuation-in-part of application No. 12/454,907, filed on May 26, 2009.
(60) Provisional application No. 61/103,601, filed on Oct. 8, 2008.
(60) Provisional application No. 61/114,503, filed on Nov. 14, 2008.

(51) Int. Cl. A61F 6/04

(52) U.S. Cl. 128/844

(57) ABSTRACT

A condom, including a non-porous sheath member, having an open end and a rounded end, and shaped to cover the glans and body portion of a penis, and at least one rib having at least one outer portion and at least one inner portion, wherein the at least one outer portion is attachedly fixed to said non-porous sheath member, and wherein an inner cylinder formed by at the at least one inner portion is exclusive from said non-porous sheath member, and including at least one lubricant within the inner cylinder and at least one hole extending from the inner cylinder through the outer portion to provide for flow of the lubricant outwardly from the inner cylinder through the at least one hole pursuant to application of a predetermined pressure, wherein the at least one rib extends at least one of substantially from the open end to the closed end and substantially about a circumference of the non-porous sheath member.

### CROSS-REFERENCE TO RELATED APPLICATIONS

This application claims priority to U.S. patent application Ser. No. 12/454,907, entitled “LUBRICATED CONDOM” and filed May 26, 2009, which claims priority to U.S. Provisional Patent Application No. 61/103,601, entitled “LUBRICATED CONDOM” filed Oct. 8, 2008, and to U.S. Provisional Patent Application No. 61/114,503, entitled “LUBRICATED CONDOM” filed Nov. 14, 2008. The entire contents of each of the aforementioned applications are incorporated herein by reference as if set forth herein in the entirety.

### FIELD OF THE INVENTION

The present invention is directed generally to a method and apparatus for providing lubrication and, more particularly, to a lubricated condom.

### BACKGROUND OF THE INVENTION

Sanitary contraceptives, such as condoms, are primarily designed to prevent the transfer of disease and unwanted pregnancy. Condoms have also been designed to provided stimulation through mechanical, chemical and visual means. Ribbed condoms that glow in the dark may provide both a mechanical an visual stimulation, for example. Further, use of external lubricants have been employed to increase satisfaction and ease of use of both the user of the condom and the user's target subject.

However, the lubrications employed historically fail to provide a sufficient amount of lubricant, and fail to provide lubrication in a timely manner. If a lubricant is only provided at the initiation of use, the lubricating effects of the provided lubricant may diminish such that no practical lubrication is provided by the condom for a period of time before use ends. This may cause irritation for the users of the condom and may cause the condom to become dislodged.

Thus, there exists a need for a condom which can provide lubrication semi-continuously and/or as desired during use, rather than providing only a limited of lubricant prior to use.

### SUMMARY OF THE INVENTION

The present invention is directed to a condom, including a non-porous sheath member, having an open end and a rounded end, and shaped to cover the glans and body portion of a penis, and at least one rib having at least one outer portion and at least one inner portion, wherein the at least one outer portion is attachedly fixed to said non-porous sheath member, and wherein an inner cylinder formed by at the at least one inner portion is exclusive from said non-porous sheath member, and including at least one lubricant within the inner cylinder and at least one hole extending from the inner cylinder through the outer portion to provide for flow of the lubricant outwardly from the inner cylinder through the at least one hole pursuant to application of a predetermined pressure, wherein the at least one rib extends at least one of substantially from the open end to the closed end and substantially about a circumference of the non-porous sheath member.

The present invention solves problems experienced with the prior art because it provides lubrication semi-continuously, and on-demand, during use. Those and other advantages and benefits of the present invention will become apparent from the detailed description of the invention hereinbelow.

### BRIEF DESCRIPTION OF THE FIGURES

The present invention will be described hereinbelow in conjunction with the following figures, in which like numerals represent like items, and wherein:

FIG. 1 illustrates an exemplary embodiment of the present invention;

FIG. 2 illustrates a cross-section of at least one rib;

FIG. 3 illustrates an exemplary embodiment of the present invention;

FIG. 4 illustrates a cross-section of the present invention;

FIG. 5 illustrates a cross-section of the present invention;

FIG. 6 illustrates a cross-section of the present invention;

FIG. 7 illustrates a cross-section of the present invention;

FIG. 8 illustrates a cross-section of a rib and a condom roll in the present invention;

FIG. 9 schematically illustrates a rolled condom in the present invention;

FIG. 10 illustrates a cross-section of the present invention;

FIG. 11 illustrates a cross-section of the present invention;

FIG. 12 illustrates a cross-section of the present invention;

FIG. 13 illustrates a cross-section of the present invention;

FIG. 14 illustrates a cross-section of the present invention;

FIG. 15 schematically illustrates a side view the present invention;

FIG. 16 illustrates a cross-section of the present invention; and

FIG. 17 schematically illustrates a side view the present invention.

### DETAILED DESCRIPTION OF THE INVENTION

It is to be understood that the figures and descriptions of the present invention have been simplified to illustrate elements that are relevant for a clear understanding of the present invention, while eliminating, for purposes of clarity, many other elements found in typical condoms. Those of ordinary skill in the art will recognize that other elements are desirable and/or required in order to implement the present invention. However, because such elements are well known in the art, and because they do not facilitate a better understanding of the present invention, a discussion of such elements is not provided herein.

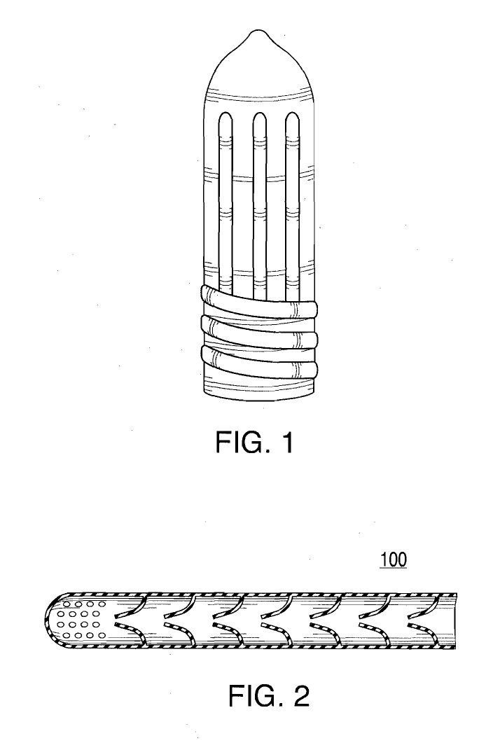

FIG. 1 illustrates a condom in accordance with the present invention. As illustrated, the condom may include two or more ribs 100 along the barrel of the condom. Such ribs may be integral to, or separate from, the length of the barrel of the condom, but in a preferred embodiment, the underlying aspects of the condom are to remain intact, watertight, and free of holes, irrespective of any holes, openings, veins, bladders, or the like integral with the aforementioned ribs of the present invention.

As used herein, the term “ribs” includes any vein, bladder, pathway, or the like, extending along any portion of the length of the barrel of a condom. As such, a rib, as used herein, provides a path for the flow of lubricant in at least a partially substantially parallel direction to the barrel of the condom, although, in certain embodiments, such flow along a rib may additionally occur along a portion substantially perpendicular to the length of the barrel of the condom.

As more particularly illustrated in FIG. 2, a rib may allow for a flow of lubricant along or around the barrel of the condom. Such flow of lubricant may, in certain preferred embodiments, be directional, i.e., one way, at least in that the rib may include veins, valves, bladders, or the like that direct the flow of lubricant, such as upward from the base of the condom toward the tip of the condom during use of the condom. Such ribs may additionally include, such as along the length of the rib or at or near a termination of a rib, an exit pathway, such as a valve, hole or holes, that allow lubricant to exit the condom. As discussed hereinabove, and in accordance with the present invention, it almost goes without saying that such valves, holes or exit pathways should in no way interact with, pierce, or otherwise allow leakage from any portion of the underlying body of the condom, thereby preventing any leakage or penile discharge from inside the condom.

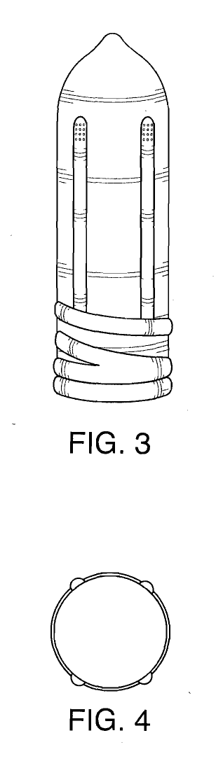

As further illustrated in FIG. 3, the ribs of the present invention may provide a pathway at the base of the condom to one or more reservoirs 200 containing the aforementioned lubricant, which may, needless to say, be spermicidal lubricant. For example, pressure exerted along the barrel of the condom and focused at the base of the condom may force lubricant out of the one or more reservoirs and along the uni-directional ribs outward toward the upper tip of the condom. Such reservoirs may simply be one, two, three, or more spirals perpendicularly around the base of the condom, filled with lubricant, and sealed at the end of the reservoir spiral closest to the base of the condom. Thereby, when force is exerted at the base of the condom, the lubricant may, in accordance with a predetermined desired flow rate, flow around the spiral reservoir or reservoirs and upward along the barrel of the condom during use. Similarly, a single reservoir may be placed laterally around or approximate to the base of the condom, and such single reservoir may or may not be connected to the aforementioned spiral ribs, wherein, upon exertion of pressure downward along the barrel of the condom toward the base, pressure is exerted on the lubricant and the lubricant is forced from the reservoir and upward through the aforementioned ribs.

More particularly, FIG. 4 illustrates a condom having multiple, discrete reservoirs. As illustrated, the multiple reservoirs may include one or more reservoirs extending latitudinally upward along the sheath of the condom, which latitudinal reservoirs may be connected to a longitudinal reservoir or reservoirs about the base of the condom sheath. Further, FIG. 4 illustrates a discrete longitudinal series of reservoirs, each extending longitudinally around the sheath, and each discrete from at least one other longitudinal reservoir, and from the latitudinal reservoirs.

At present, the use of condoms presents various difficulties remedied by the present invention. For example, in prior art embodiments, lubrication provided on the condom may be providing in quantities greater than necessary in order to provide initial lubrication. Thereby, excess lubrication may reach the inside of the condom, such as when the condom is rolled up, and may thus cause the condom to be loose or difficult to use. This may particularly be the case when, in use, additional lubrication is added to the exterior of the condom. For example, such additional lubrication may simply work its way down to the base of the condom and have little effect during usage. As will be apparent to those skilled in the art in light of the discussion herein, the present invention remedies such issues. For example, the reserve lubrication provided in the present invention may be, in effect, time released, such as based on the presence of veins or valves as discussed herein, and may be released at the position most in need of lubrication during use of the condom, that is, at the tip of the condom. As such, the present invention may be particularly useful in remedying the dryness that typically occurs after longer periods of usage of a condom. Likewise, the time release or targeted release of lubrication in the condom of the present invention may be prevent buildup of lubrication in undesired areas, that is, in those areas that may cause slippage of the condom, or difficulty in unrolling the condom.

Needless to say, the present invention may be provided with multiple reserves of lubricant, such as wherein each reservoir is separately time released, or is subject to release under different pressures, or subject to simultaneous release. Such multiple reservoirs may be used to create, for example, a ribbed effect for additional pleasure during use, as well as providing additional targeted lubricant during the use of the present invention.

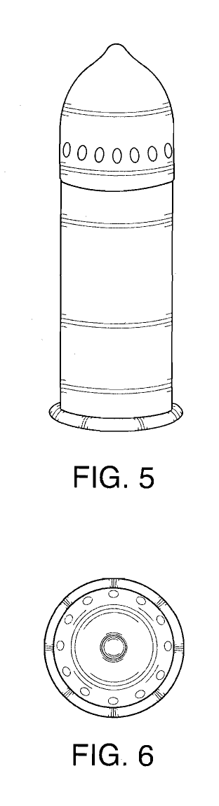

In addition to and alternatively to the veins and/or tubular reservoirs for delivering lubricant as discussed hereinabove, FIG. 5 illustrates a cross-sectional view of a condom having an extended spermicidal reservoir tip, as is known in the art, and additionally having a lubricant reservoir above the prior art extended tip. More particularly, FIG. 5 illustrates a condom sheath atop which is a prior art “extended tip”, such as for improved containing of the force of an ejaculation, and a lubricant reservoir above or associated with the prior art extended tip. The lubricant discussed herein may be delivered from this upper reservoir. As illustrated in the cross-sectional view of FIG. 6, the reservoir may deliver lubricant via one or more holes, valves, or the like, as discussed above with respect to FIGS. 1-4, and may deliver the lubricant responsive to pressure, breakage, physical removal of impediments to lubricant flow, expansion, or the like.

For example, and particularly with respect to the embodiments illustrated in FIGS. 1, 3, 5-7, and 10-17, the lubricant may be released by a pressure, such as may be applied to the condom by manual squeezing, thrusting with the condom, or the like. Further, breakage of thin seals located, for example, atop the lubricant reservoir of FIGS. 5 and 6, such as by use of the condom or unrolling of the condom, may allow for the release of the lubricant. Yet further, physical removal of, for example, a seal placed on the uppermost portion of the condom, such as while the condom is rolled and/or within its packaging, may be removed in order to allow release of the lubricant. Finally, pressure caused by expansion, such as of the penis within the condom, may effectuate release of the lubricant.

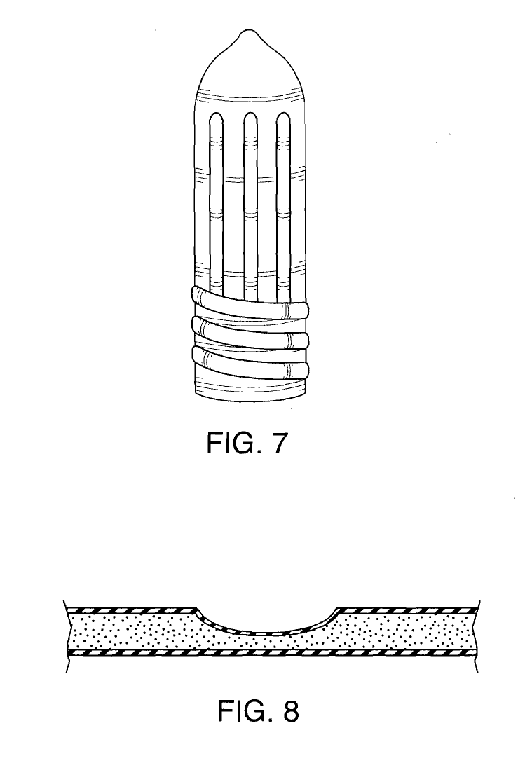

FIG. 7 illustrates, in the manner discussed hereinabove, that one or more lubricant reservoir ringed ribs may be placed about the condom. As discussed above, and in preferred embodiments, the lubricant ring ribs may be distinct from the underlying continuous layer of the condom, such that bodily fluids released within the condom cannot leak into the lubricant ring and/or through the lubricant ring to the exterior of the condom. The lubricant ring ribs illustrated in FIG. 7 may remain in an overlapped position as to one another of the multiple ring ribs, such as while the condom is in its rolled and/or packaged configuration.

FIG. 8 illustrates a lubricant ring rib of the present invention, wherein the condom roll is peripherally and circumferentially outside the lubricant ring rib in the rolled position. As illustrated, the lubricant ring rib includes at least one hole that is laterally adjacent to the condom roll at the outer periphery of the lubricant ring rib. This at least one hole at the outermost portion of the lubricant ring rib, may, as illustrated in FIG. 8, be plugged prior to unrolling of the condom. Such a plug used prior to the unrolling of the condom may include, for example, the inverted nipple illustrated in FIG. 8, wherein the inverted nipple is substantially aligned with, and plugs the flow of lubricant from, the at least one hole in the lubricant ring rib. In embodiments wherein the inverted nipple or a similar plug is provided by the condom roll, it will be apparent to those skilled in the art that the unrolling of the condom for use will remove the plug from the hole in the lubricant ring rib, thereby allowing for the release of lubricant. Of course, it may be further appreciated that, as discussed hereinthroughout, removal of the plug may not lead to immediate release of the lubricant, but rather the lubricant may remain in situ due to the presence of one or more valves, veins, thin seals, or the like, atop the hole.

It will be further appreciated, particularly in light of the discussion herein with respect to FIG. 8, that the condom roll and ring rib of the condom of the present invention may not only serve to provide a plug for lubricant flow, but additionally may be provided in various locations, or multiple locations, along the shaft of the sheath of the condom, such as to suppress blood flow and thereby enhance performance, and/or to maintain position of the condom during use. More particularly, a known manner of unrolling the condom may be provided, wherein the known roll provides an outer roll, and wherein the rib(s) of the present invention provide a second, or inner, roll that may provide lubricant, plug lubricant, and/or provide an additional pressure ring about the condom.

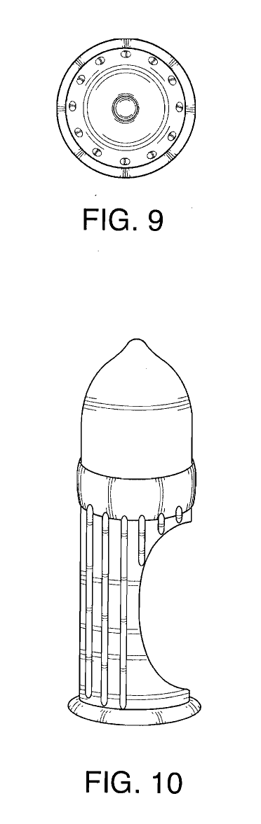

For example, FIG. 9 illustrates an embodiment of the present invention wherein a ring rib of lubricant is provided about the circumference of the condom, and wherein the roll of the condom is provided about the outermost circumference of the lubricant ring about. In the illustrated embodiment, the outer roll of the condom may provide adhesive and/or the aforementioned inverted nipple as a plug for the lubricant. When the condom is unrolled, the lubricant may exit the lubricant ring rib, and further either or both of the lubricant ring rib and the condom roll may be latitudinally moved up or down the shaft of the penis.

Thereby, and as more particularly illustrated in FIG. 10, variations of known condoms may be provided that improve the sexual experience, but that do not increase the likelihood of leakage of bodily fluids. For example, FIG. 10 illustrates the lubricant ring rib of the present invention holding the condom in place at a point approximately halfway down the unrolled condom. This ring rib may provide lubricant, and may additionally be used, as illustrated at the base of the unrolled condom in FIG. 10, in conjunction with the prior art roll to further hold the condom in place. It will be noted that the exemplary condom of FIG. 10 includes openings for direct stimulation of the penis latitudinally along the sheath of the condom between the lubricant ring rib and the roll, and these openings may be provided because sufficient constrictive pressure is available to hold the condom in place and prevent bodily fluid leakage based on the presence of the lubricant ring rib halfway down the condom shaft.

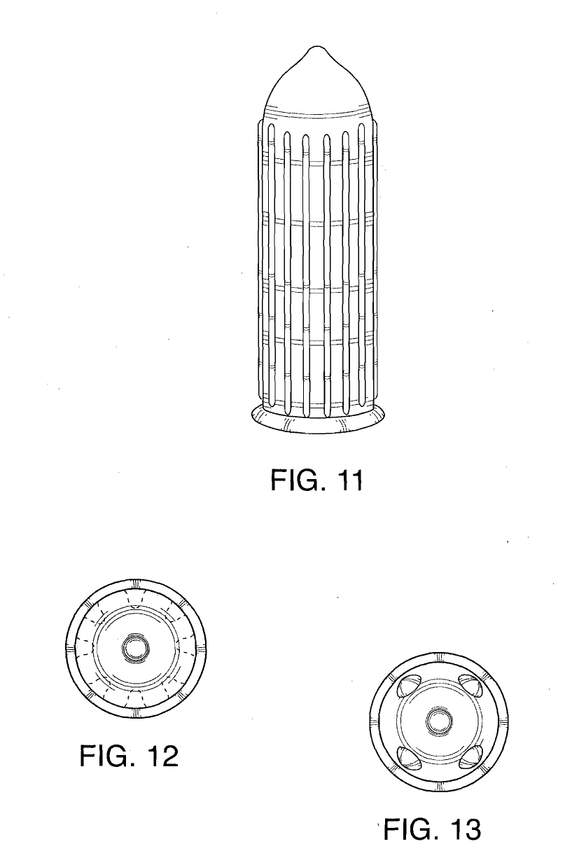

FIG. 11 illustrates an additional variation of the lubricant ring rib of the present invention functioning to provide additional pressure to hold the condom in place over and above the prior art condom roll. More specifically, FIG. 11 illustrates the use of vertical reservoir ribs to provide pressure to affix the condom along the penis, as opposed to the horizontal reservoir rib illustrated in certain of the exemplary embodiments hereinabove.

FIGS. 12 and 13 illustrate alternative embodiments of horizontal cross sections of the vertical reservoir rib embodiment of FIG. 11. More particularly, FIG. 12 illustrates a cross section of a condom in which the lubricant reservoirs are provided distinct from the condom walls, but inside the outer circumference of the condom. Thereby, the outer circumference of the condom may be substantially circular, but within the circle of the condom's periphery may be lubricant reservoirs that apply additional pressure on the penis within the condom. Nevertheless, the lubricant may be released from these vertical reservoirs by the same means as have been discussed hereinthroughout. Similarly to FIG. 12, FIG. 13 illustrates a cross section wherein the vertical reservoir ribs are outside of a periphery of the condom walls.

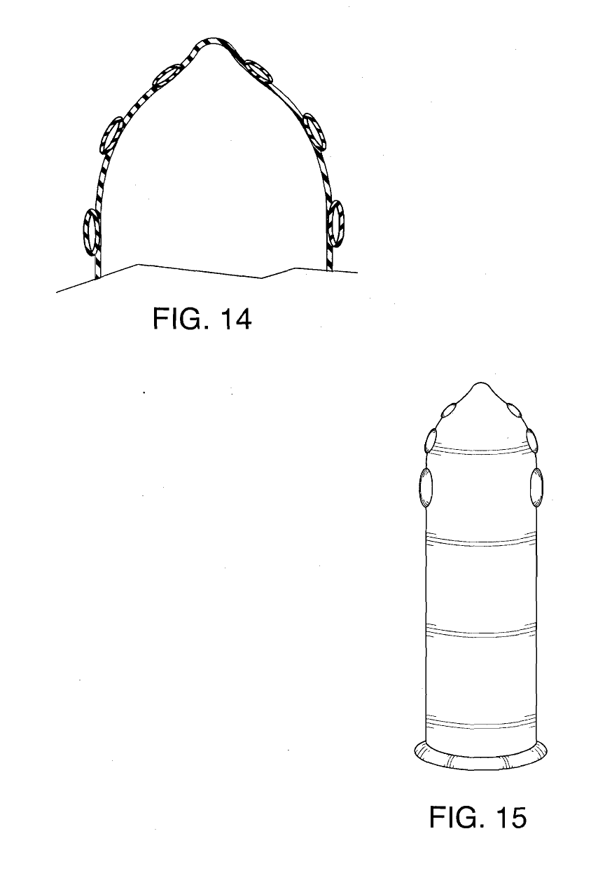

Those skilled in the art will appreciate, in light of the discussion herein, that various additional means may be used in order to force lubricant from, or block lubricant within, the lubricant reservoir ribs. Exemplary ones of these means are illustrated in FIGS. 14, 15, 16 and 17.

More particularly, FIG. 14 illustrates a series of reservoir ribs for lubricant, wherein ones of the series of ribs have differing sizes, heights, lengths, widths, or the like. Variations in pressure along these reservoir ribs of varying size will, if the reservoir ribs include valves, veins, or seals, for example, force lubricant from the reservoir ribs at different times. For example, lubricant may begin to exit the larger reservoir ribs first, and may later exit the smaller reservoir ribs as the larger reservoir ribs empty and the pressure on the smaller reservoirs consequently increases.

FIG. 15 illustrates with particularity that reservoir ribs may be smaller in size, and may be provided as a series of lubricant filled bump ribs along the shaft of the condom. Thereby, as various parts of the condom are inserted, and/or as the pressure upon various parts of the condom increases, different ones of the lubricant bump ribs may be squeezed, may tear, or may pop, and may thereby release the lubricant.

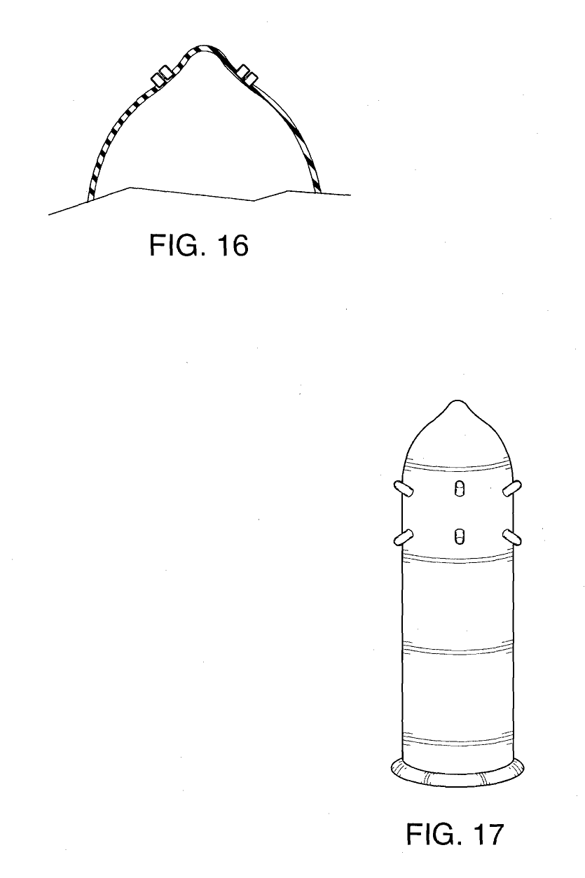

FIG. 16 illustrates a two-stage lubricant reservoir rib having a thin seal between the two stages. As illustrated, when pressure is applied to the two-stage reservoir rib, the thin seal will tear, thus releasing lubricant from each stage of the reservoir rib. It will be appreciated by those skilled in the art that this two-stage reservoir rib may have different constituents in each stage, and that the tearing of the seal may thus allow for a mixing of the constituents, such as a mixing of lubricant and spermicide.

FIG. 17 illustrates an embodiment similar to that of FIG. 15, but the embodiment of FIG. 17 illustrates that the lubricant bump ribs may be directionally formed. Thereby, when a force is applied in the opposing phase of the direction of the bump rib, the bump rib may be pulled back, may tear at its base, and lubricant may be consequently released. Of course, the directional bump ribs may be formed in different directions along the same condom shaft, such that certain of the bump ribs may release lubricant upon inward thrust, and others of the bump ribs may release lubricant upon pulling outward. Further, in embodiments such as those discussed above, variations in the size and/or pressure reactivity of the bump ribs may, as illustrated in FIG. 17, allow for release of lubricant both at variable times, and with variable directions of thrust.

Those skilled in the art will appreciate that the embodiments discussed herein provide a significant improvement in the sexual experience. For example, the pressure-based release of lubricant provided in the present invention may allow for release of lubricant on-demand, such as by manual squeezing, thereby preventing interruption of the sexual experience in order to obtain lubrication, and thereby further preventing too much friction or not enough friction during the sexual experience.

The disclosure herein is directed to the variations and modifications of the elements and methods of the invention disclosed that will be apparent to those skilled in the art in light of the disclosure herein. Thus, it is intended that the present invention covers the modifications and variations of this invention, provided those modifications and variations come within the scope of the appended claims and the equivalents thereof.

We claim:

1. A condom, comprising:
a non-porous sheath member, having an open end and shaped to cover the glans and body portion of a penis;
at least one rib extending around said non-porous sheath member and having an outer portion and an inner portion, wherein the inner portion is exclusive from said non-porous sheath member, and comprising:
at least one lubricant between the inner portion and the outer portion; and
a plurality of holes along the outer portion from which the lubricant exits responsive to pressure;
at least one roll about a periphery formed at least by the outer portion and comprising a plurality of inverted nipples substantially aligned with the plurality of holes to at least substantially preclude the exit of the lubricant until an unrolling of said at least one roll removes the plurality of inverted nipples from the alignment with the plurality of holes.

2. The condom of claim 1, wherein said rib is substantially filled with said lubricant.

3. The condom of claim 1, wherein said rib further comprises at least one valve wherein said valve prevents the exit of the lubricant.

4. The condom of claim 3, wherein said valve is within the outer portion.

5. The condom of claim 1, wherein said pressure is applied externally to said rib.

6. The condom of claim 1, wherein said sheath member comprises a thermoplastic elastic material.

7. The condom of claim 1, wherein said sheath member comprises a thermoplastic polyurethane elastomer.

8. The condom of claim 1, wherein said rib comprises a thermoplastic elastic material.

9. The condom of claim 1, wherein said rib comprises a thermoplastic polyurethane elastomer.

10. The condom of claim 1, wherein said sheath member comprises a material selected from the group consisting of: polyurethanes; polyether block amides; styrene-rubber-styrene block copolymers; polyesters; olefinic homopolymers and copolymers; and copolymers, composites and blends thereof.

11. The condom of claim 1, wherein said rib comprises a material selected from the group consisting of: polyurethanes; polyether block amides; styrene-rubber-styrene block copolymers; polyesters; olefinic homopolymers and copolymers; and copolymers, composites and blends thereof.

13. The condom of claim 1, wherein the open end has a varying diameter penis receiving compartment.

14. The condom of claim 1, wherein said sheath member is watertight.

15. The condom of claim 1, wherein said at least one rib is patterned to provide stimulation during intercourse.

16. The condom of claim 1, wherein said sheath member additionally comprises a secondary lubricant coating.

17. A condom, comprising:
a non-porous sheath member having an extended spermicidal reservoir tip, an open end, and being shaped to cover the glans and body portion of a penis;
at least one lubricant reservoir extending away from said non-porous sheath member proximate to the reservoir tip at an opposing surface of said non-porous sheath member from a surface contacting the penis; and
a plurality of exit valves atop said at least one lubricant reservoir, wherein lubricant within said lubricant reservoir is released from ones of said plurality of exit valves responsively to application of pressure to said non-porous sheath member at least at the spermicidal extended tip.

18. The condom of claim 17, wherein said sheath member comprises a material selected from the group consisting of: polyurethanes; polyether block amides; styrene-rubber-styrene block copolymers; polyesters; olefinic homopolymers and copolymers; and copolymers, composites and blends thereof.

19. The condom of claim 17, wherein said lubricant reservoir comprises a material selected from the group consisting of: polyurethanes; polyether block amides; styrene-rubber-styrene block copolymers; polyesters; olefinic homopolymers and copolymers; and copolymers, composites and blends thereof.

20. A condom, comprising:
a non-porous sheath member having an open end, and being shaped to cover the glans and body portion of a penis; and
a plurality of lubricant-filled bumps along said non-porous sheath member, wherein at least first ones of said plurality have an acute angle of alignment with the open end, and wherein at least second ones of said plurality have an obtuse angle of alignment with the open end;
wherein, upon application of pressure along said sheath member toward the open end, the second ones at least partially release the lubricant, and upon application of pressure along said sheath member away from the open end, the first ones at least partially release the lubricant.

21. The condom of claim 20, wherein said sheath member is watertight.
